(19) United States
(12) Patent Application Publication
(10) Pub. No.: US 2024/0293249 A1
(43) Pub. Date: Sep. 5, 2024

(54) RESTRAINT SYSTEM

(71) Applicant: NOEL CHEN, SAN FRANCISCO, CA (US)

(72) Inventor: NOEL CHEN, SAN FRANCISCO, CA (US)

(21) Appl. No.: 18/519,446

(22) Filed: Nov. 27, 2023

Related U.S. Application Data
(60) Provisional application No. 63/488,377, filed on Mar. 3, 2023.

(51) Int. Cl. A61F 5/37; A47C 21/02

(52) U.S. Cl. CPC A61F 5/3776; A47C 21/022

(57) ABSTRACT

A bedsheet restraint system is provided to securely hold a person in place. The restraint system restrains body parts such as limbs, wrists, ankles, torso, elbows and knees to a bedsheet carried by a bed. Body parts are slidably placed through and held in place by the restraining sleeves until desired to be removed.

### RELATED APPLICATION

This application claims the benefit of provisional patent application Ser. No. 63/488,377, filed 3 Mar. 2023.

### BACKGROUND OF THE INVENTION

The present invention relates to a restraint system for restraining a subject to a bedsheet carried by a bed. Prior art structures use restraints to keep toddlers from rolling over in their sleep, to restrain prisoners, and to restrain unruly people during surgery.

Hospital bed restraints are used to secure patients in place to prevent them from falling out of bed, to protect them from harm, or to prevent them from interfering with medical procedures.

In some cases, patients may be physically or cognitively unable to control their movements, and bed restraints provide a way to keep them safe and prevent injury. For example, a patient with a head injury may be at risk of falling out of bed, and bed restraints can be used to prevent this from happening.

In other cases, patients may have a medical condition or be under the influence of medications that cause them to become restless or combative, and bed restraints can be used to prevent them from interfering with medical procedures or harming themselves or others. For example, a patient undergoing a surgical procedure may need to be restrained to prevent them from moving during the procedure.

The use of bed restraints in hospitals is regulated by law and is subject to ethical considerations. Bed restraints are usually only used when necessary and in accordance with established protocols and guidelines, and patients should be monitored regularly while they are restrained. Additionally, bed restraints should be used in a way that minimizes discomfort and harm to the patient.

In another usage scenario, restraints can be used in sexual bondage, which refers to the practice of consensual restraint or confinement of one person by another, typically as part of a sexual or BDSM (bondage, dominance, sadism, and masochism) activity. Bondage often involves the use of ropes, handcuffs, blindfolds, or other restraints to restrict a person's movement and heighten sensory experiences.

The origins of bondage can be traced back to ancient civilizations, where forms of restraint were used for both practical and ceremonial purposes. Today, bondage is often practiced as part of BDSM relationships and may involve various levels of pain, sensation play, and power exchange. In these situations, participants may build trust in their relationships.

Bondage should be conducted consensually, with clear communication and agreement between all parties involved. Additionally, it is recommended to use caution and proper safety measures to minimize the risk of injury or harm.

Bondage is a personal and intimate practice that may vary greatly between individuals and couples. Some may view it as a way to explore power dynamics, while others may see it as a form of artistic expression. Regardless of the reasons, bondage is a form of sexuality that has been gaining increased recognition and acceptance in recent years.

Restraint systems of the prior art are designed such that it is difficult for the subject to restrain himself or herself. Additionally, restraint systems of the prior art restrain typically wrists and ankles.

### SUMMARY OF THE INVENTION

A bedsheet restraint system designed to securely hold a person in place while allowing another person to easily access the first person's body, for example for medical procedures or for sexual bondage is disclosed. The restraint system of the present invention restrains body parts such as wrists, ankles, torso, and limbs including elbows and knees. The present invention preferably provides access to the torso, armpits and private parts of a subject.

The bedsheet restraint system of the present invention is designed to provide a safe and secure way to hold subjects in place during medical procedures, or participants in sexual activity in place during that activity. The bedsheet restraint system comprises a bedsheet made of a soft and comfortable material, and sleeve restraints that can be secured, permanently or removably, to the bedsheet. The bedsheet is itself removably secured to a bed, so the bedsheet keeps the subject secure to the bedsheet, and thus the bed. The bedsheet and sleeve restraints are made of materials that are safe and gentle on the subject's skin, and the system is adjustable to accommodate subjects of different sizes.

The sleeve restraints are sized and configured to keep the subject secure, but not too tight as to cause discomfort, obstruct blood circulation or restrict breathing. The sleeve restraints are also made of a soft and comfortable material to minimize discomfort.

The bedsheet restraint system is easy to use and can be quickly adjusted to fit different subject sizes. Medical staff can easily access the subject's body for medical procedures while the subject remains securely in place. The system is also easy to clean and maintain, making it ideal for use.

In a preferred embodiment, a particular experience of stability and comfort is provided by coupling sleeves preferably made of elastic fabric (for comfort and to accept different limb sizes), to a bedsheet. The sleeves can be attached to the bedsheet for instance by adhesives, hook and loop material, zippers, or by sewing. A securing mechanism (retaining straps), such as hook and loop fastening, an elastic fastening, or garter grip fastening, is coupled to the bedsheet, and provided for securing the bedsheet itself about the bed (preferably underneath the bed) for securing the bedsheet in place. The bedsheet can be provided with further hook and loop material or garter grip fastening, to provide for adjustability of the length and width of the bedsheet. Alternatively, the bedsheet itself, and the sleeves, may comprise at least one of an elastic (retractable) fabric, inelastic fabric, waterproof fabric, a combination of the foregoing (e.g., a waterproof layer under the breathable fabric for comfort purposes), or materials such as vinyl fabric, leather, plastic, spandex, neoprene, rubber, etc.

In use, when a subject views the present invention, the subject shall have a clear sense that he/she will easily to escape, and therefore reduce fear. This differs from when subjects see the restraint beds in the prior art, which may themselves cause the subject resisting to get into the restraint due to a fear that they are not able to escape. In use of the present invention, the subject may be able move himself/herself into or out of the bedsheet sleeves.

Hook and loop straps or adjustable straps with garter grip on one or both ends can be employed to stabilize the bedsheet in place, which is particularly useful when the subject is struggling. If garter grips are used, they may be attached to nylon (or fabric, rope) straps with adjustable mechanisms such as slide buckles.

In an additional embodiment, comfortable wrist and ankle cuffs can be coupled with a soft pad large enough that the subject cannot retract limbs through the sleeves coupled to the bedsheet.

### BRIEF DESCRIPTION OF DRAWINGS

FIG. 1 is a perspective in-use view of a bedsheet restraint system of the present invention;

FIG. 2 is a side view thereof;

FIG. 2A is an end view thereof;

FIG. 3 is a close-up side view of an overfold of the bedsheet used for changing height (or width) dimensions of the bedsheet;

FIG. 4 is a top in-use view of one embodiment of a sleeve restraint used on an arm;

FIGS. 5-8A are various top in-use views of different arrangements of sleeve restraints;

FIGS. 9A-9C are perspective top, bottom, and side views respectively, of one embodiment of a wrist and ankle cuff;

FIG. 9D is a perspective view of a wrist and ankle cuff in a folded condition;

FIG. 9E is a top view of the alternate embodiment of a wrist and ankle cuff shown in FIGS. 9A-9D, worn by a user whose arm is through a sleeve restraint carried by a bedsheet;

FIGS. 9F and 9G are side views of an alternate embodiment of a wrist and ankle cuff carrying an appendage arrangement, respectively, for optional use with the bedsheet restraint system of the present invention;

FIG. 10 is a perspective in-use view of a bedsheet restraint system of the present invention, with optional wrist and ankle cuffs and appendages worn by a user.

### DETAILED DESCRIPTION OF THE INVENTION

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims. In no event should a user, wearer or subject of the present invention be considered a positively claimed limitation to the claims.

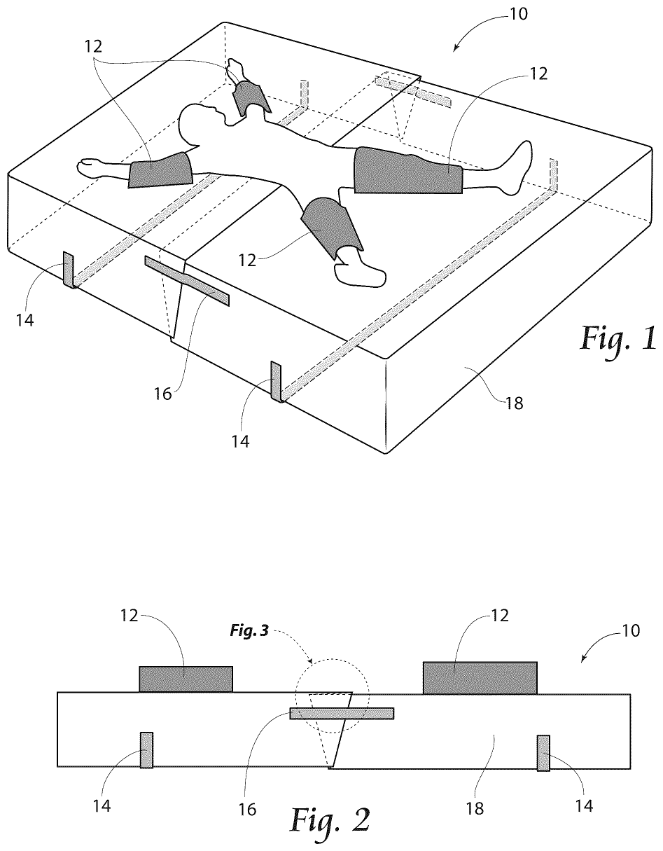

Referring now to FIG. 1, a perspective in-use view of a bedsheet restraint system 10 of the present invention is shown. A bedsheet 18 is shown which may be placed around a mattress (the mattress not shown) as is well known. One or a series of retaining straps 14 can be used to secure the bedsheet 18 about the mattress. The retaining straps 14 can comprise fabric or any elastic material to accommodate different mattress sizes. A series of sleeves or sleeve restraints 12 are coupled to a top surface of the bedsheet 18. The sleeve restraints 12 are sized and configured to preferably receive arms, legs and optionally torsos of a user, who is pictured in FIG. 1.

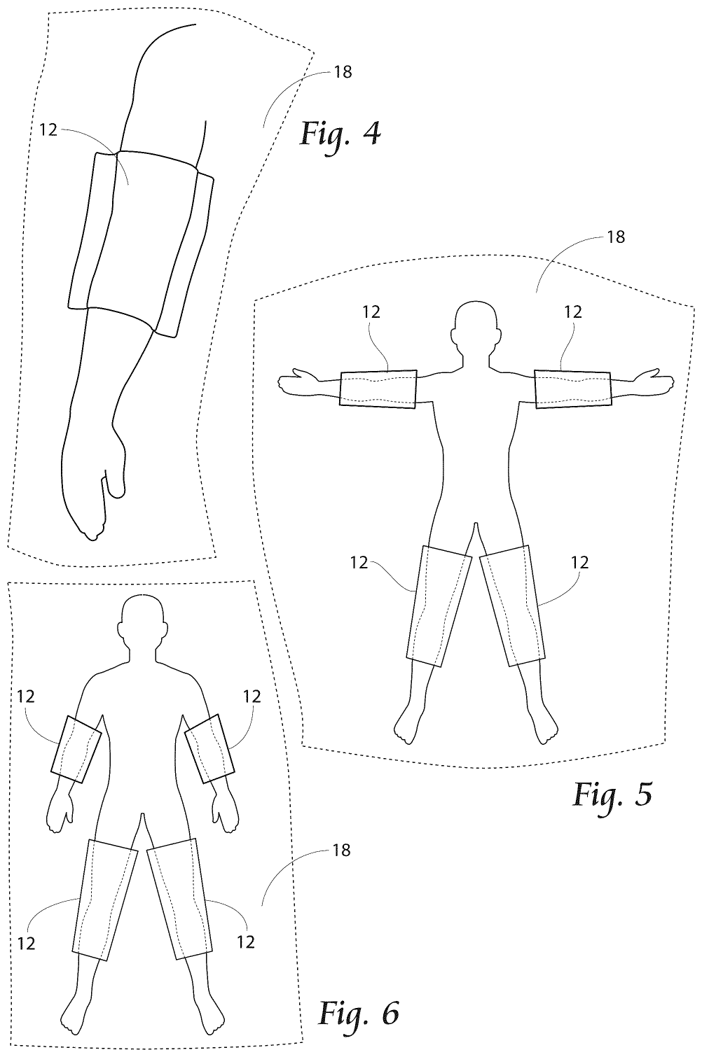

Sleeve restraints 12 may be formed of a flat sheet of any suitable material (rubber, fabrics including flannel, linen, lyocell, microfiber, polyester, sateen, percale, bamboo, etc.) which may be elasticized fabric, as shown later with reference to FIG. 4, or may be formed by a tube of suitable material. Sleeve restraints 12 may be coupled to the bedsheet 18 for instance by sewing, by zippers, adhesive, or by hook and loop material (which allows for different in-use configurations of sleeve restraints 12).

At the side of the bedsheet 18, an optional side strap 16 is shown, as will be described later.

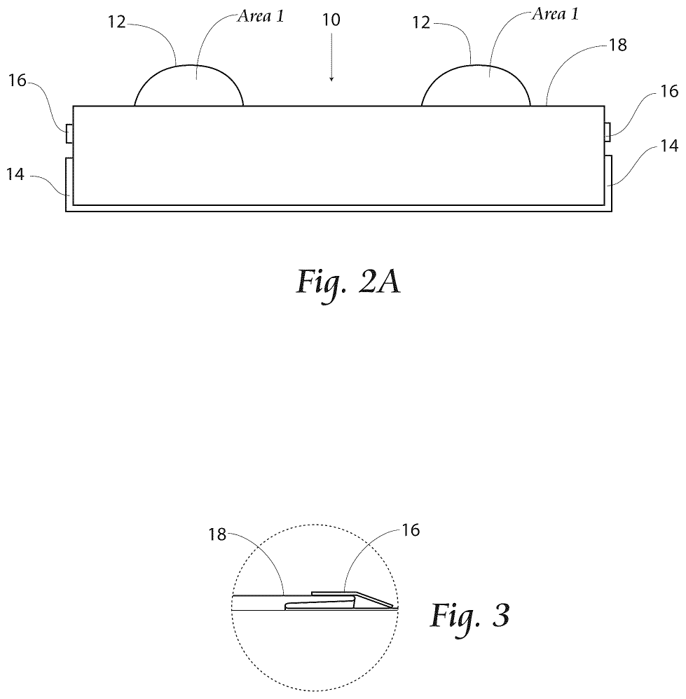

Referring now to FIGS. 2 and 2A, a side and an end view, respectively, of bedsheet restraint system 10 is shown. In the illustrated embodiment, two sleeve restraints 12 are shown, although as described later, any combination or arrangement of sleeve restraints 12 is within the scope of the invention.

Referring to FIG. 2A, an area between sleeve restraints 12 and bedsheet 18 has a cross-sectional area of Area 1, which may be a variable area if elasticized material is used to form sleeve restraints (but, to a maximum area based on stretch capabilities of the material).

Referring to FIG. 3, side strap 16 is used to keep in place an optional overfold of the bedsheet 18. Side strap 16 is coupled to the bedsheet 18, preferably detachably at least one end of side strap 16. The purpose of the optional overfold of bedsheet 18 is so that the length or width of bedsheet 18 can be shortened to accommodate different length or width mattresses (e.g., single, queen, king etc.). Also in this manner the position of sleeve restraints 12 relative to the edge of the bedsheet 18 can be varied to accommodate subjects of different sizes. The overfold may be kept in place by securing a selectively free-end of side strap 16 to bedsheet 18 (e.g., by hook and loop material, garter grips snaps, buttons, or the like, not shown). Similarly, two side straps 16 can be used to adjust both the length and the width of the bedsheet 18 (not shown). Side strap 16 may preferably detachable on two initially free ends, or adjustment of length or width of bedsheet 18 may be with a side strap with one free and one fixed end, such as by using a garter grip.

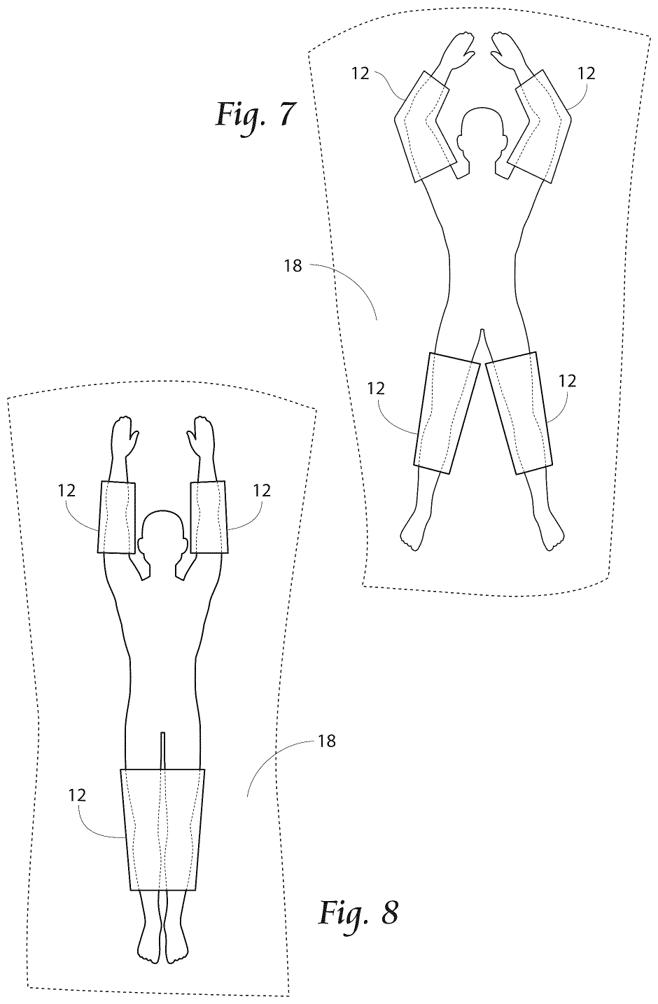

Referring now to FIG. 4, a top in-use view of one embodiment of a sleeve restraint 12 used on an arm is shown. In FIGS. 4-8 and 9E that follow, only a portion of bedsheet 18 is shown. In an optional embodiment, sleeve restraint 12 is configured to restrain arm, and preferably elbow movement (or leg/knee movement in the case of a knee restraint 12), and sleeve restraint 12 can either be straight as shown in FIG. 4, or bent as shown in FIG. 7.

Referring now to FIGS. 5-8, various top in-use views of different arrangements of sleeve restraints 12 are shown. It is not necessary to couple any specific number of sleeve restraints 12 to one bedsheet 18. Several different combinations of sleeve restraints 12 can be provided in different poses, which can be coupled to the same bedsheet 18, providing flexibility to choose which pose is used when the bedsheet 18 is in use. As can be seen, sleeve restraints 12 can be placed for different arm and leg securement arrangements. A subject can use the restraint system either face up (as shown in the Figures) or face down (not shown).

For exemplary purposes, if four sleeve restraints are coupled to the bedsheet 18, in one preferred arrangement in addition to a position depicted in FIG. 1, both arms and legs of a subject are extended when in use, as shown in FIG. 5. In that arrangement, sleeve restraints 12 for arm placement can be collinear as shown in FIG. 5. In another preferred arrangement, a left arm sleeve restraint 12 can be parallel to a left leg sleeve restraint 12, and a right arm sleeve restraint 12 can be parallel to a right leg sleeve restraint 12, as shown in FIG. 6. In yet another preferred arrangement, right and left arm sleeve restraints 12 can be bent to allow for hands to be placed above a head as shown in FIG. 7. In yet another preferred arrangement, right and left arm sleeve restraints 12 can be straight and parallel to allow for hands to be placed above a head as shown in FIG. 8. FIG. 8 also shows a larger sleeve restraint 12, sized to receive both legs of a subject.

A single sleeve restraint 12 can also be used to constrain both legs (FIG. 8), or a single sleeve such as shown in FIG. 8 can be sized relatively larger and placed to constrain a torso (not shown). Additionally, variations and combinations of the number and positioning of sleeve restraints 12 can be used. For instance, a series of two, three or more pairs of sleeve restraints 12 can be provided at once, for both different arm and leg positioning (e.g, providing a plurality of pairs of sleeve restraints 12 for the arms on a single bedsheet 18 to accommodate a plurality of arm positions such as raised, out to the side, or lowered). Any combination of the foregoing is also within the scope of the invention.

As shown in FIG. 8A, any number of sleeve restraints 12 can be provided in different arrangements on bedsheet 18, or accommodations to rearrange sleeve restraints 12 in infinite fixed or movable arrangements.

Further, if a detachable arranged is chosen for coupling sleeve restraints 12 to bedsheet 18, different arrangements of sleeve restraints 12 can be provided by providing different landing zones on bedsheet 18, for instance by providing several individual areas of hook or loop material to bedsheet 18, each capable of receiving sleeve restraints 12 in different positions (not shown). For instance, hook or loop material landing zones can be coupled to bedsheet 18 for receiving sleeve restraints 12 both for accommodating arms in an extended manner as shown in FIG. 5, as well as accommodating arms in a hands above head manner as shown in FIG. 8. One bedsheet 18 not only can include different arm positions, two or more different leg positions (FIG. 7 and FIG. 8), but also be arranged on the same bedsheet 18. In this manner, a single bedsheet 18 can accommodate sleeve restraints 12 in many different positions.

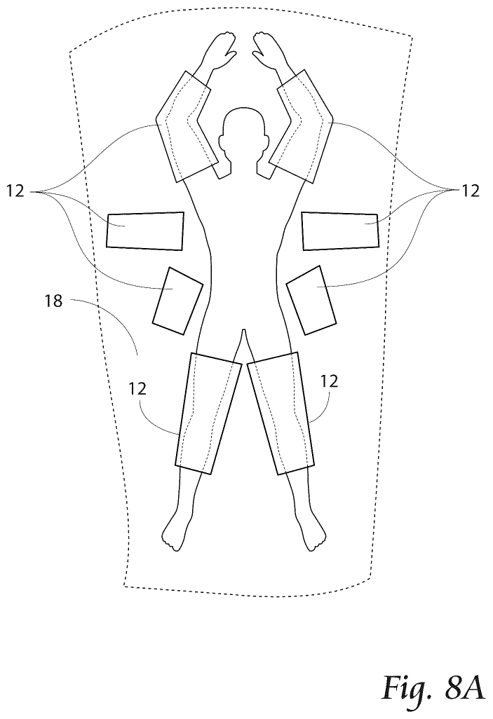

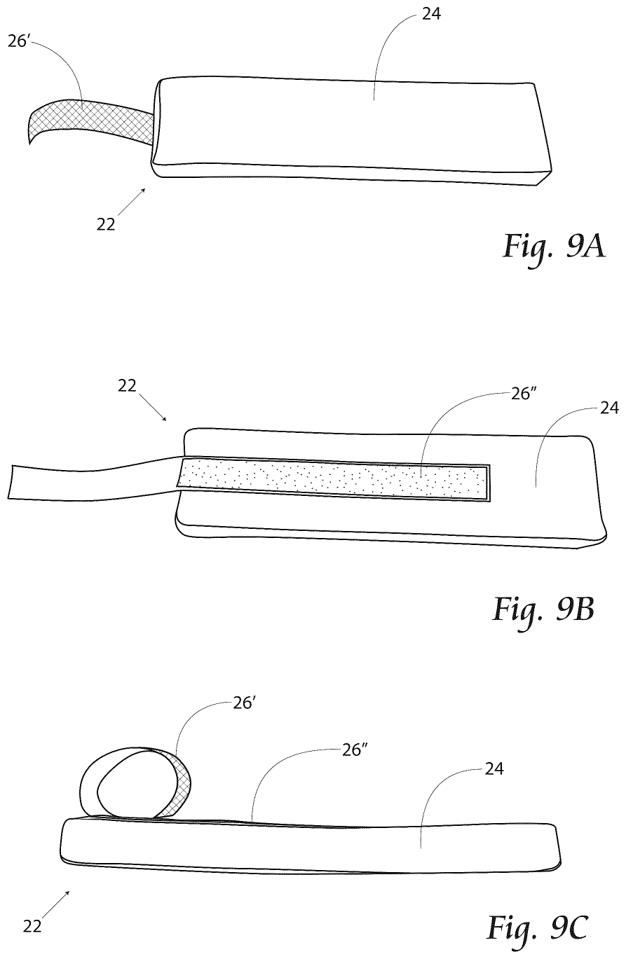

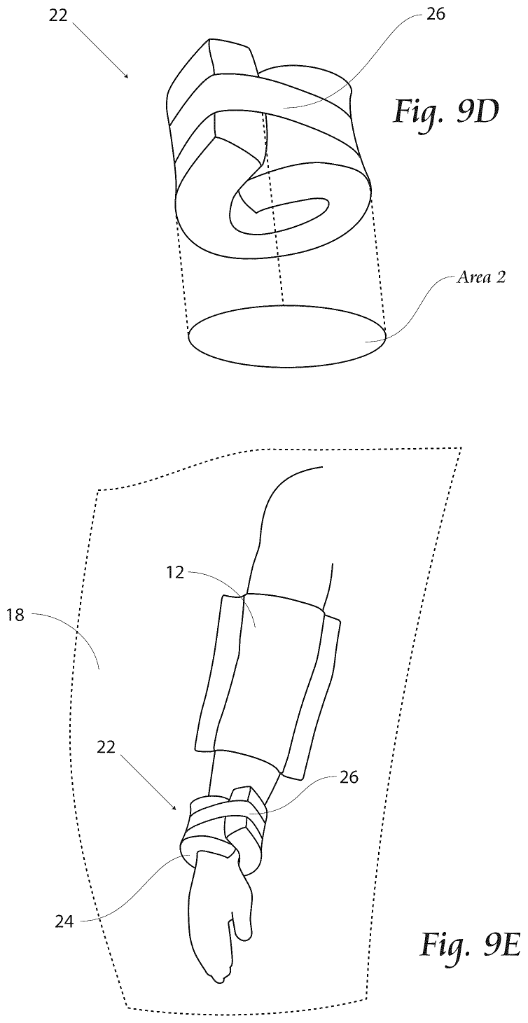

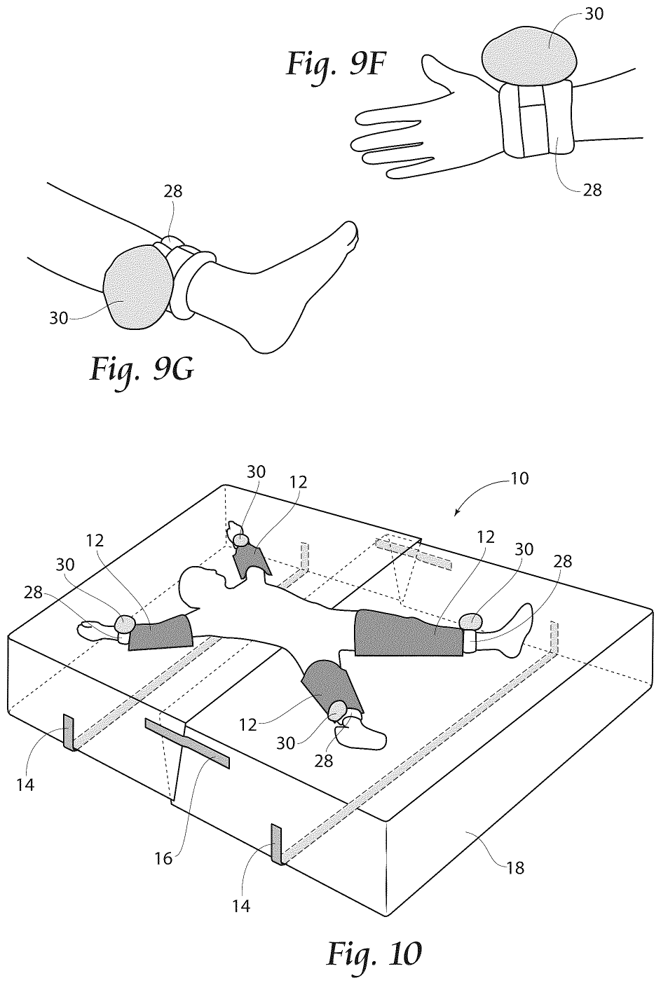

Referring now to FIGS. 9A-9C, perspective top, bottom, and side views respectively, of a wrist or ankle cuff 22 are shown. Cuff 22 comprises a segment of preferably relatively thick softer and flexible material 24 carrying a strip of hook and loop fastener 26 material (hook 26′, loop 26″). Flexible material 24 having greater than 1 cm thickness is preferred, and in a more preferred embodiment, flexible material 24 has a thickness between 1 cm and 15 cm is preferably used, more preferably between 1 cm and 15 cm, although thinner material 24 may of course be used. The strip of hook and loop fastener 26 material is coupled to material 24 for instance by sewing, adhesives, heat molding, or the like.

Referring now to FIG. 9D, a perspective view of wrist or ankle cuff 22 in a folded condition is shown, with wrist or ankle cuff 22 having a cross-sectional area of Area 2.

Referring now to FIG. 9E, wrist and ankle cuff 22 is shown worn by a subject whose arm is through a sleeve restraint 12 carried by bedsheet 18. In use, flexible material 24 can be folded and constrained about a body part of a subject, such as a wrist as shown in FIG. 9E, by attaching hook material 26′ to loop material 26″. The size and thickness of flexible material 24, when worn about a body part such as the wrist or ankle, prevents the wrist or ankle from easy slidable disengagement from sleeve 12. This is because the effective cross-sectional area of a combination of the subject's body part and the wrist or ankle cuff 22, forming Area 2 (FIG. 9D), is larger than the area between sleeve restraints 12 and bedsheet 18 having a cross-sectional area of Area 1 (see FIG. 2A). In this way, the user is securely constrained within sleeves 12 (when desired), as the arms and legs are not as easily withdrawn from sleeve restraints 12.

Referring now to FIGS. 9F and 9G, side views of an alternate embodiment of a wrist and ankle cuff 28 and appendage 30 arrangement for optional use with the bedsheet restraint system 10 of the present invention are shown, worn about a wrist or ankle of a user similar to cuff 22 described previously. In one embodiment, appendage 30 comprises a ball-shaped object or obstruction 30 coupled to cuff 28, for instance by sewing, adhesive, or hook and loop material. Obstruction 30 can be manufactured with materials both soft and firm, for instance materials such as those used to produce sponges, racquetballs, tennis balls, golf balls, plastic balls, or the like. In a preferred embodiment, cuff 28 is sized to fit about a wrist or ankle of a user, and appendage 30 is sized to project and extend outwardly from the cuff 28 in order to increase the cross-sectional area of cuff 28 alone.

In use, as shown in FIG. 10, a user can wear a cuff 28 about either one or more wrists, ankles or both, and the cuff 28 carries appendage 30. The combined size of cuff 28 and appendage 30, when worn about a wrist or ankle, selectively prevents the wrist or ankle from easy slidable disengagement from sleeves 12. In this embodiment, the user is securely constrained within sleeves 12 (when desired), as the arms and legs are not as easily withdrawn from sleeve restraints 12.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. A restraint system comprising:
a sheet of material;
a first restraining sleeve coupled to said sheet of material in a fixed orientation, said first restraining sleeve comprising a first open end and a second open end, said second open end having a first cross-sectional area;
a second restraining sleeve coupled to said sheet of material in a fixed orientation, said second restraining sleeve comprising a third open end and a fourth open end.

2. A restraint system according to claim 1, said first restraining sleeve and said second restraining sleeve formed of an elasticized fabric.

3. A restraint system according to claim 1, said first restraining sleeve configured to receive therethrough a first body appendage of a user, and said second restraining sleeve configured to receive therethrough a second body appendage of said user.

4. A restraint system according to claim 1, said first restraining sleeve and said second restraining sleeve each individually coupled to said sheet of material.

5. A restraint system according to claim 1, said sheet of material comprising a length dimension and a width dimension, said sheet of material carrying a retaining strap in said width dimension, and carrying a side strap in said length dimension.

6. A restraint system according to claim 5, said side strap detachably coupled to said sheet of material on at least a first end of said side strap, said side strap configured to fix said length dimension of said sheet of material at a first length, and a second length shorter than said first length.

7. A restraint system according to claim 1, said kit further comprising a third restraining sleeve and a fourth restraining sleeve, said first sleeve substantially collinear with said third sleeve.

8. A restraint system according to claim 1, said first sleeve substantially parallel to said second sleeve.

9. A restraint system according to claim 1, said first sleeve substantially parallel with said third sleeve.

10. A restraint system according to claim 1, said first sleeve bent.

11. A kit comprising said restraint system of claim 1 and a cuff having a second cross-sectional area greater than said first cross-sectional area of said second open end.

12. A kit according to claim 11, said cuff carrying an appendage, said appendage increasing said second cross-sectional area of said cuff.

13. A kit according to claim 11, said cuff formed with a material thickness greater than 1 cm.

14. A kit according to claim 11, said cuff formed with a material thickness between 1 cm and 10 cm.

15. A restraint system comprising:
a sheet of material;
a first restraining sleeve coupled to said sheet of material, said first restraining sleeve comprising a first open end and a second open end, said second open end having a first cross-sectional area;
a second restraining sleeve coupled to said sheet of material, said second restraining sleeve comprising a third open end and a fourth open end;
said sheet of material comprising a plurality of first restraining sleeve landing zones for detachably coupling said first restraining sleeve to said sheet of material, to allow placement of said first restraining sleeve in a plurality of orientations.

16. A restraint system according to claim 15, said sheet of material comprising a plurality of second restraining sleeve landing zones for detachably coupling said second restraining sleeve to said sheet of material, to allow placement of said second restraining sleeve in a plurality of orientations.
